(19) United States
(12) Patent Application Publication
(10) Pub. No.: US 2024/0350594 A1
(43) Pub. Date: Oct. 24, 2024

(54) METHODS AND COMPOSITIONS FOR TREATING HYPERGLYCEMIA AND DIABETES

(71) Applicant: Anagram Therapeutics, Inc., Framingham, MA (US)

(72) Inventors: Alexey Margolin, Newton, MA (US); Stefan Pierzynowski, Trelleborg (SE); Kateryna Pierzynowska, Trelleborg (SE)

(21) Appl. No.: 18/683,423

(22) PCT Filed: Aug. 12, 2022
(86) PCT No.: PCT/US2022/074932

§ 371 Date: Feb. 13, 2024

Related U.S. Application Data
(60) Provisional application No. 63/232,920, filed on Aug. 13, 2021.

(51) Int. Cl. A61K 38/44; A61K 9/00; A61P 3/10

(52) U.S. Cl. CPC A61K 38/443; A61K 9/0053; A61K 38/44; A61P 3/10; C12Y 101/03004; C12Y 111/01006

(57) ABSTRACT

Disclosed are pharmaceutical compositions comprising a glucose oxidase enzyme and a peroxide-degrading enzyme (e.g., a catalase enzyme), which can be used, among other things, to treat hyperglycemia, diabetes, obesity, metabolic syndrome, and/or an eating disorder.

### CROSS-REFERENCE TO RELATED APPLICATIONS

This application claims the benefit of and priority to U.S. Provisional Patent Application No. 63/232,920, filed on Aug. 13, 2021, the disclosure of which is hereby incorporated by reference in its entirety for all purposes.

### FIELD OF THE INVENTION

The invention relates generally to methods and compositions for treating hyperglycemia, diabetes, obesity, metabolic syndrome, and/or an eating disorder, and more particularly, the invention relates to compositions containing a glucose oxidase enzyme and a peroxide-degrading enzyme (e.g., a catalase enzyme) and their use in treating hyperglycemia, diabetes, obesity, metabolic syndrome, and/or an eating disorder.

### BACKGROUND

According to the Centers for Disease Control (CDC) 34.2 million people in the US have diabetes and 88 million people or more than every third adult have prediabetes. Total medical cost and lost work and wages for people with diagnosed diabetes amount to $327 billion. The diabetes epidemic is not limited to the US. According to the World Health Organization (WHO), the number of adults living with diabetes has almost quadrupled since 1980 to 422 million adults. Diabetes is a major cause of blindness, kidney failure, heart attack, stroke, and lower limb amputation. Almost half of all deaths attributable to high blood glucose occur before the age of 70 years. WHO estimates that diabetes was the seventh leading cause of death in 2016. According to the World Federation of Diabetes, 4 million people die every year from diabetes.

It has been shown that aggressive management of blood glucose levels can dramatically reduce the diabetes mortality. However, despite proven benefits of glycemic control nearly half of the population does not meet glycemic goals. Carbohydrate-rich foods with high glycemic index and glycemic load, in particular, contribute to elevated postprandial glucose (PPG), and carbohydrate absorption from intestinal lumen can, in turn, contribute to the development of chronic diseases such as obesity and diabetes (Vlachos et al. (2020) Nutrients 12 (6): 1561).

Accordingly, there is an ongoing need for new and effective pharmaceutical compositions and therapies for regulating carbohydrate absorption and treating hyperglycemia, diabetes, obesity, and/or metabolic syndrome.

### SUMMARY OF THE INVENTION

The invention is based, in part, of the discovery of pharmaceutical compositions comprising a glucose oxidase enzyme and a peroxide-degrading enzyme (e.g., a catalase enzyme) that can be used to treat hyperglycemia and/or diabetes in a subject in need thereof.

Accordingly, in one aspect, the invention provides a pharmaceutical composition comprising (or consisting essentially of) a glucose oxidase enzyme, a peroxide-degrading enzyme, and a pharmaceutically acceptable excipient.

In certain embodiments, the glucose oxidase enzyme is spray-dried or lyophilized. In certain embodiments, the glucose oxidase enzyme is a microbial glucose oxidase enzyme, or a functional fragment or variant thereof. In certain embodiments, the glucose oxidase enzyme is derived from Aspergillus niger, for example, the glucose oxidase enzyme comprises SEQ ID NO: 1, SEQ ID NO: 2, or a functional fragment or variant thereof. In certain embodiments, the pharmaceutical comprises from about 1,000 to about 250,000 international units (I.U.) of the glucose oxidase enzyme.

In certain embodiments, the peroxide-degrading enzyme is spray-dried or lyophilized. In certain embodiments, the peroxide-degrading enzyme is a catalase enzyme or a peroxidase enzyme, e.g., the peroxide-degrading enzyme is a catalase enzyme, which can be a microbial catalase enzyme, or a functional fragment or variant thereof. In certain embodiments, the catalase enzyme is derived from Aspergillus niger, for example, the catalase enzyme comprises SEQ ID NO: 3, SEQ ID NO: 4, or a functional fragment or variant thereof. In certain embodiments, the pharmaceutical comprises from about 1,000 to about 250,000 international units (I.U.) of the catalase enzyme. Depending upon the circumstances, in the composition, the ratio of international units of glucose oxidase to international units of the peroxide-degrading enzyme is in the range from 0.1 to 10.

In certain embodiments, the composition is formulated as an oral dosage form. In certain embodiments, the composition is formulated as a solid oral dosage form, for example, a powder, satchel, granulate, pellet, micropellet, tablet, or minitablet. In certain embodiments, the composition is formulated as a liquid oral dosage form, for example, an elixir, syrup, or drop. In certain embodiments, the composition is formulated as a nanoparticle or a nanopreparation.

In certain embodiments, the composition has a shelf-life at room temperature of at least 3 months, 6 months, 9 months, 12 months, 15 months, 18 months, 21 months, 24 months, 36 months, 48 months, 60 months, 72 months, 84 months, 96 months, 108 months, or 120 months.

In certain embodiments, the composition does not include a glucoamylase enzyme, a sucrase enzyme, a lactase enzyme, a transglucosidase enzyme, an α-amylase enzyme or a combination thereof.

In another aspect, the invention provides a method of treating diabetes (e.g., type 2 diabetes) in a subject in need thereof. The method comprises administering (for example, orally administering) to the subject an effective amount of any of the enzymes or compositions described herein, to treat diabetes in the subject.

In another aspect, the invention provides a method of treating obesity in a subject in need thereof. The method comprises administering (for example, orally administering) to the subject an effective amount of any of the enzymes or compositions described herein, to treat obesity in the subject.

In another aspect, the invention provides a method of treating metabolic syndrome in a subject in need thereof. The method comprises administering (for example, orally administering) to the subject an effective amount of any of the enzymes or compositions described herein, to treat metabolic syndrome in the subject.

In another aspect, the invention provides a method of treating hyperglycemia in a subject in need thereof. The method comprises administering (for example, orally administering) to the subject an effective amount of any of the enzymes or compositions described herein, to treat hyperglycemia in the subject.

In another aspect, the invention provides a method of treating hyperinsulinemia in a subject in need thereof. The method comprises administering (for example, orally administering) to the subject an effective amount of any of the enzymes or compositions described herein, to treat hyperinsulinemia in the subject.

In another aspect, the invention provides a method of treating an eating disorder (such as bulimia or carbohydrate hyperphagia) in a subject in need thereof. The method comprises administering (for example, orally administering) to the subject an effective amount of any of the enzymes or compositions described herein, to treat the eating disorder in the subject.

In another aspect, the invention provides a method of reducing a level of glucose in a subject in need thereof. The method comprises administering (for example, orally administering) to the subject an effective amount of any of the enzymes or compositions described herein, to reduce the level of glucose in the subject. The method may reduce the level of glucose in the blood and/or gastrointestinal tract of the subject, or may reduce the level of glucose in a sample from the subject (e.g., a body fluid sample, e.g., a blood, serum or plasma sample).

In certain embodiments, the method reduces the level of glucose in the blood of the subject from 0 to 240 minutes or 0 to 360 minutes following a meal or snack, as determined by an Area Under Curve (AUC) analysis, by at least about 10%, 20%, 30%, 40%, 50%, 60%, 70%, 80%, or 90%. In certain embodiments, the method reduces glucose Cmax in the blood of the subject following a meal or snack by at least about 10%, 20%, 30%, 40%, 50%, 60%, 70%, 80%, or 90%. In certain embodiments, the method reduces glucose Tmax in the blood of the subject following a meal or snack by at least about 10%, 20%, 30%, 40%, 50%, 60%, 70%, 80%, or 90%. In certain embodiments, when administered together with a food, the method reduces the glycemic index of the food by at least about 10%, 20%, 30%, 40%, 50%, 60%, 70%, 80%, or 90%. In certain embodiments, when administered together with a food, the method reduces the caloric intake of the food by at least about 10%, 20%, 30%, 40%, 50%, 60%, 70%, 80%, or 90%.

In another aspect, the invention provides a method of reducing a level of C-peptide and/or insulin in a subject in need thereof. The method comprises administering (e.g., orally administering) to the subject an effective amount of any of the enzymes or compositions described herein, to reduce the level of C-peptide and/or insulin in the subject. The method may reduce the level of C-peptide and/or insulin in the blood and/or gastrointestinal tract of the subject, or may reduce the level of C-peptide and/or insulin in a sample from the subject (e.g., a body fluid sample, e.g., a blood, serum or plasma sample). In certain embodiments, the method reduces the level of C-peptide and/or insulin in the blood of the subject from 0 to 240 minutes or 0 to 360 minutes following a meal or snack, as determined by an Area Under Curve (AUC) analysis, by at least about 10%, 20%, 30%, 40%, 50%, or 60%.

In another aspect, the invention provides a method of reducing a level of HAlc in a subject in need thereof. The method comprises administering (e.g., orally administering) to the subject an effective amount of any of the enzymes or compositions described herein, to reduce the level of HAlc in the subject. The method may reduce the level of HAlc in the blood of the subject, or may reduce the level of HAlc in a sample from the subject (e.g., a body fluid sample, e.g., a blood, serum or plasma sample).

In another aspect, the invention provides a method of reducing a level of glycemic load of the consumed carbohydrates-rich diet. The method comprises administering (for example, orally administering) to the subject an effective amount of any of the enzymes or compositions described herein, to reduce the level of glucose in the GI tract available for absorption.

In certain embodiments of any of the foregoing methods, the subject has diabetes, obesity, metabolic syndrome, hyperglycemia, hyperinsulinemia, and/or an eating disorder (such as bulimia or carbohydrate hyperphagia).

Depending upon the circumstances, the enzyme or composition can be administered to the subject 1, 2, 3, 4, or more than 4 times per day. Furthermore, the enzyme or composition can be administered to the subject together with a meal or snack (e.g., at each meal or snack).

These and other aspects and features of the invention are described in the following detailed description and claims.

### DESCRIPTION OF THE DRAWINGS

The invention can be more completely understood with reference to the following drawings.

FIGS. 1A-1B depict glucose absorption curves during Meal Glucose Tolerance Test (MGTT) IV (including enzyme treatment with 450 mg glucose oxidase and 0.8 ml catalase; black line (boxes)) compared to MGTT III (with no enzyme treatment, black line (circles)). FIG. 1A depicts glucose absorption following a meal of feed only. FIG. 1B depicts glucose absorption following a meal of feed and glucose powder. All data points correspond to mean±SD, AUCs are presented as mean±SEM.

FIG. 2 depicts glucose absorption curves during a Meal Glucose Tolerance Test (MGTT) V, which included a group receiving enzyme treatment with 900 mg glucose oxidase and 1.6 ml catalase (“Treatment”) and a group with no enzyme treatment (“Control”). All data points correspond to mean±SEM, AUCs are presented as mean±SEM.

FIG. 3 depicts glucose absorption curves during Meal Glucose Tolerance Test (MGTT) VI, which included a group receiving a meal of feed only (“Control Feed”), a group receiving a meal of feed and glucose powder (“Control Feed+Glucose”), and a group receiving a meal of feed and glucose powder as well as enzyme treatment with 450 mg glucose oxidase and 0.8 ml catalase (“Treatment Feed+Glucose”). All data points correspond to mean±SD, AUCs are presented as mean±SEM.

FIGS. 4A-4B depict C-peptide release curves during Meal Glucose Tolerance Test (MGTT) IV (including enzyme treatment with 450 mg glucose oxidase and 0.8 ml catalase, black line (boxes)) compared to MGTT III (with no enzyme treatment, black line (circles)). FIG. 4A depicts C-peptide release following a meal of feed only. FIG. 4B depicts C-peptide release following a meal of feed and glucose powder. All data points correspond to mean±SD. AUCs are presented as mean±SEM.

FIG. 5A depicts C-peptide release curves during Meal Glucose Tolerance Test (MGTT) V, which included a group receiving enzyme treatment with 900 mg glucose oxidase and 1.6 ml catalase (“MGTT V Treatment”, black line (boxes)) and a group with no enzyme treatment (“MGTT V Control” black line (circles)). FIG. 5B depicts a comparison of C-peptide release curves during MGTT V, MGTT IV (including enzyme treatment with 450 mg glucose oxidase and 0.8 ml catalase, Group 1), and MGTT III (with no enzyme treatment, Group 1). All data points correspond to mean±SD.

FIG. 6 depicts C-peptide release curves during Meal Glucose Tolerance Test (MGTT) VI, which included a group receiving a meal of feed only (“Control Feed”), a group receiving a meal of feed and glucose powder (“Control Feed+Glucose”), and a group receiving a meal of feed and glucose powder as well as enzyme treatment with 450 mg glucose oxidase and 0.8 ml catalase (“Treatment Feed+Glucose”). All data points correspond to mean±SD.

### DETAILED DESCRIPTION

The invention is based, in part, upon the discovery of pharmaceutical compositions comprising a glucose oxidase enzyme and a peroxide-degrading enzyme (e.g., a catalase enzyme) that can be used to treat diabetes (e.g., type 2 diabetes), hyperglycemia, hyperinsulinemia, obesity, and metabolic syndrome in a subject in need thereof. Various features and aspects of the invention are discussed in detail below.

### I. Enzymes

Among other things, the invention provides pharmaceutical compositions including a glucose oxidase enzyme and a peroxide-degrading enzyme that, for example, are useful in treating disorders such as diabetes (e.g., type 2 diabetes), hyperglycemia, hyperinsulinemia, obesity, and metabolic syndrome.

As used herein, the term “glucose oxidase” refers to any enzyme, or a functional fragment thereof, that is capable of catalyzing the oxidation of β-D-glucose to D-glucono-ω-lactone and hydrogen peroxide and/or the conversion of D-glucono-ω-lactone to gluconic acid. Glucose oxidase typically catalyzes at least the following reaction:

β-D-glucose+O₂→D-glucono-ω-lactone+H₂O₂.

Glucose oxidase is also referred to as EC 1.1.3.4, glucose oxyhydrase, corylophyline, penatin, glucose aerodehydrogenase, microcid, β-D-glucose oxidase, D-glucose oxidase, D-glucose-1-oxidase, β-D-glucose: quinone oxidoreductase, glucose oxyhydrase, and deoxin-1, and, unless indicated otherwise, the terms are used interchangeably herein. The term glucose oxidase includes variants having one or more amino acid substitutions, deletions, or insertions relative to a wild-type glucose oxidase sequence, and/or fusion proteins or conjugates including a glucose oxidase. As used herein, the term “functional fragment” of a glucose oxidase refers to fragment of a full-length glucose oxidase that retains, for example, at least 10%, at least 20%, at least 30%, at least 40%, at least 50%, at least 60%, at least 70%, at least 80%, at least 90%, or 100% of the enzymatic activity of the corresponding full-length, naturally occurring glucose oxidase. Glucose oxidase enzymatic activity may be assayed by any method known in the art. Exemplary glucose oxidase activity assays are available from Sigma-Aldrich (Cat. No. MAK097).

Exemplary glucose oxidase enzymes include glucose oxidase enzymes derived from Aspergillus niger. Amino acid sequences of exemplary wild-type glucose oxidase enzymes derived from Aspergillus niger are depicted in SEQ ID NO: 1 (with signal sequence) and SEQ ID NO: 2 (without signal sequence). Additional exemplary glucose oxidase enzymes can be found on the world wide web at brenda-enzymes.org/enzyme.php?ecno=1.1.3.4#ORGANISM.

As used herein, the term “peroxide-degrading enzyme” refers to any enzyme, or a functional fragment thereof, that is capable of degrading hydrogen peroxide. Exemplary peroxide-degrading enzymes include catalase and peroxidase enzymes.

As used herein, the term “catalase” refers to any enzyme, or a functional fragment thereof, that is capable of catalyzing the decomposition of hydrogen peroxide to water and oxygen. Catalase typically catalyzes the following reaction:

$$2 {H}_{2} {O}_{2} \to {O}_{2} + 2 {H}_{2} O$$

Catalase is also referred to as EC 1.11.1.6, equilase, caperase, optidase, catalase-peroxidase, and CAT, and, unless indicated otherwise, the terms are used interchangeably herein. The term catalase includes variants having one or more amino acid substitutions, deletions, or insertions relative to a wild-type catalase sequence, and/or fusion proteins or conjugates including a catalase. As used herein, the term “functional fragment” of a catalase refers to fragment of a full-length catalase that retains, for example, at least 10%, at least 20%, at least 30%, at least 40%, at least 50%, at least 60%, at least 70%, at least 80%, at least 90%, or 100% of the enzymatic activity of the corresponding full-length, naturally occurring catalase. Exemplary catalase activity assays are available from Sigma-Aldrich (Cat. Nos. CAT100 and 219265).

Exemplary catalase enzymes include catalase enzymes derived from Aspergillus niger. Amino acid sequences of exemplary wild-type catalase enzymes derived from Aspergillus niger are depicted in SEQ ID NO: 3 (with signal sequence) and SEQ ID NO: 4 (without signal sequence). Additional exemplary catalase enzymes can be found on the world wide web at brenda-enzymes.org/enzyme.php?ecno=1.11.1.6#ORGANISM.

As used herein, the term “peroxidase” refers to any enzyme, or a functional fragment thereof, that is capable of catalyzing an oxidation-reduction reaction by the mechanism of a free radical that can transform a compound into an oxidized or polymerized product. Peroxidases typically catalyze the following reaction:

$${H}_{2} {O}_{2} + {A H}_{2} \to 2 {H}_{2} O + A$$

The term peroxidase includes variants having one or more amino acid substitutions, deletions, or insertions relative to a wild-type peroxidase sequence, and/or fusion proteins or conjugates including a peroxidase. As used herein, the term “functional fragment” of a peroxidase refers to fragment of a full-length peroxidase that retains, for example, at least 10%, at least 20%, at least 30%, at least 40%, at least 50%, at least 60%, at least 70%, at least 80%, at least 90%, or 100% of the enzymatic activity of the corresponding full-length, naturally occurring peroxidase. Exemplary peroxidase activity assays are available from Sigma-Aldrich (Cat. No. MAK092).

Peroxidases represent a large group of enzymes. Exemplary peroxidase enzymes can be found on the world wide web at brenda-enzymes.org/enzyme.php?ecno=1.11.1.7#ORGANISM.

In certain embodiments, the glucose oxidase and/or peroxide-degrading enzyme comprise at least one (for example, one, two, three, four, five, six, seven, or eight) mutation(s) relative to a wild type glucose oxidase and/or peroxide-degrading enzyme disclosed herein.

In certain embodiments, the glucose oxidase and/or peroxide-degrading enzyme comprise one or more conservative substitutions relative to a glucose oxidase and/or peroxide-degrading enzyme disclosed herein. In other embodiments, the glucose oxidase and/or peroxide-degrading enzyme comprises one or more non-conservative substitutions relative to a glucose oxidase and/or peroxide-degrading enzyme disclosed herein. As used herein, the term “conservative substitution” refers to a substitution with a structurally similar amino acid. For example, conservative substitutions may include those within the following groups: Ser and Cys; Leu, Ile, and Val; Glu and Asp; Lys and Arg; Phe, Tyr, and Trp; and Gln, Asn, Glu, Asp, and His. Conservative substitutions may also be defined by the BLAST (Basic Local Alignment Search Tool) algorithm, the BLOSUM substitution matrix (e.g., BLOSUM 62 matrix), or the PAM substitution: p matrix (e.g., the PAM 250 matrix). Non conservative substitutions are amino acid substitutions that are not conservative substitutions.

In certain embodiments, a disclosed glucose oxidase and/or peroxide-degrading enzyme (or a pharmaceutical composition, e.g., a solid pharmaceutical composition, comprising the same) has a shelf-life (e.g., retains at least 30%, at least 40%, at least 50%, at least 60%, at least 70%, at least 80%, at least 85%, at least 90%, at least 95%, at least 96%, at least 97%, at least 98%, at least 99%, or 100% of its biological activity) at room temperature of at least 3 months, at least 6 months, at least 9 months, at least 12 months, at least 15 months, at least 18 months, at least 21 months, at least 24 months, at least 36 months, at least 48 months, at least 60 months, at least 72 months, at least 84 months, at least 96 months, at least 108 months, or at least 120 months. In certain embodiments, a disclosed glucose oxidase and/or peroxide-degrading enzyme (or a pharmaceutical composition, e.g., a solid pharmaceutical composition, comprising the same) may have a shelf-life at room temperature of from about 3 to about 120 months, from about 3 to about 96 months, from about 3 to about 72 months, from about 3 to about 48 months, from about 3 to about 24 months, from about 3 to about 21 months, from about 3 to about 18 months, from about 3 to about 15 months, from about 3 to about 12 months, from about 3 to about 9 months, from about 3 to about 6 months, from about 6 to about 120 months, from about 6 to about 96 months, from about 6 to about 72 months, from about 6 to about 48 months, from about 6 to about 24 months, from about 6 to about 21 months, from about 6 to about 18 months, from about 6 to about 15 months, from about 6 to about 12 months, from about 6 to about 9 months, from about 9 to about 120 months, from about 9 to about 96 months, from about 9 to about 72 months, from about 9 to about 48 months, from about 9 to about 24 months, from about 9 to about 21 months, from about 9 to about 18 months, from about 9 to about 15 months, from about 9 to about 12 months, from about 12 to about 120 months, from about 12 to about 96 months, from about 12 to about 72 months, from about 12 to about 48 months, from about 12 to about 24 months, from about 12 to about 21 months, from about 12 to about 18 months, from about 12 to about 15 months, from about 15 to about 120 months, from about 15 to about 96 months, from about 15 to about 72 months, from about 15 to about 48 months, from about 15 to about 24 months, from about 15 to about 21 months, from about 15 to about 18 months, from about 18 to about 120 months, from about 18 to about 96 months, from about 18 to about 72 months, from about 18 to about 48 months, from about 18 to about 24 months, from about 18 to about 21 months, from about 21 to about 120 months, from about 21 to about 96 months, from about 21 to about 72 months, from about 21 to about 48 months, from about 21 to about 24 months, from about 24 to about 120 months, from about 24 to about 96 months, from about 24 to about 72 months, from about 24 to about 48 months, from about 48 to about 120 months, from about 48 to about 96 months, from about 48 to about 72 months, from about 72 to about 120 months, from about 72 to about 96 months, or from about 96 to about 120 months.

Glucose oxidase and/or peroxide-degrading enzyme stability, shelf-life, or half-life may be measured by any method known in the art, including enzymatic activity or SDS-PAGE analysis following incubation of an enzyme at selected conditions (e.g., temperature, pH, and/or humidity conditions) for a selected time period. It is understood that glucose oxidase and/or peroxide-degrading enzyme stability, shelf-life, or half-life will depend upon the experimental conditions in which it is measured.

In certain embodiments, the glucose oxidase and/or peroxide-degrading enzyme has at least 80%, 85%, 90%, 95%, 96%, 97%, 98%, or 99% sequence identity to a glucose oxidase and/or peroxide-degrading enzyme disclosed herein. For example, in certain embodiments, the glucose oxidase enzyme has at least 80%, 85%, 90%, 95%, 96%, 97%, 98%, or 99% sequence identity to SEQ ID NO: 1 or SEQ ID NO: 2. In certain embodiments, the peroxide-degrading enzyme has at least 80%, 85%, 90%, 95%, 96%, 97%, 98%, or 99% sequence identity to SEQ ID NO: 3 or SEQ ID NO: 4. Sequence identity may be determined in various ways that are within the skill in the art, e.g., using publicly available computer software such as BLAST, BLAST-2, ALIGN or Megalign (DNASTAR) software. BLAST (Basic Local Alignment Search Tool) analysis using the algorithm employed by the programs blastp, blastn, blastx, tblastn and tblastx (Karlin et al., (1990) PROC. NATL. ACAD. SCI. USA 87:2264-2268; Altschul, (1993) J. MOL.EVOL. 36, 290-300; Altschul et al., (1997) NUCLEIC ACIDS RES. 25:3389-3402, incorporated by reference) are tailored for sequence similarity searching. For a discussion of basic issues in searching sequence databases, sec Altschul et al., (1994) NATURE GENETICS 6:119-129, which is fully incorporated by reference. Those skilled in the art can determine appropriate parameters for measuring alignment, including any algorithms needed to achieve maximal alignment over the full length of the sequences being compared. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) PROC. NATL. ACAD. SCI. USA 89:10915-10919, fully incorporated by reference). Four blastn parameters may be adjusted as follows: Q=10 (gap creation penalty); R=10 (gap extension penalty); wink=1 (generates word hits at every wink.sup.th position along the query); and gapw=16 (sets the window width within which gapped alignments are generated). The equivalent Blastp parameter settings may be Q=9; R=2; wink=1; and gapw=32. Searches may also be conducted using the NCBI (National Center for Biotechnology Information) BLAST Advanced Option parameter (e.g.: -G, Cost to open gap [Integer]: default=5 for nucleotides/11 for proteins; −E, Cost to extend gap [Integer]: default=2 for nucleotides/1 for proteins; -q, Penalty for nucleotide mismatch [Integer]: default=-3; -r, reward for nucleotide match [Integer]: default=1; -e, expect value [Real]: default=10; -W, wordsize [Integer]: default=11 for nucleotides/28 for megablast/3 for proteins; -y, Dropoff (X) for blast extensions in bits: default=20 for blastn/7 for others; -X, X dropoff value for gapped alignment (in bits): default=15 for all programs, not applicable to blastn; and -Z, final X dropoff value for gapped alignment (in bits): 50 for blastn, 25 for others). ClustalW for pairwise protein alignments may also be used (default parameters may include, e.g., Blosum62 matrix and Gap Opening Penalty=10 and Gap Extension Penalty=0.1). A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

It is contemplated that a disclosed glucose oxidase and/or peroxide-degrading enzyme may be modified, engineered or chemically conjugated. For example, it is contemplated that a disclosed glucose oxidase and/or peroxide-degrading enzyme can be conjugated to an effector agent using standard in vitro conjugation chemistries. If the effector agent is a polypeptide, the glucose oxidase and/or peroxide-degrading enzyme can be chemically conjugated to the effector or joined to the effector as a fusion protein. Construction of fusion proteins is within ordinary skill in the art.

In certain embodiments, depending upon a particular mode of administration or site of activity, a disclosed glucose oxidase and/or peroxide-degrading enzyme can be modified with a moiety that improves its stabilization and/or retention in circulation, e.g., in blood, serum, or other tissues. For example, a disclosed glucose oxidase and/or peroxide-degrading enzyme may be conjugated to a polymer, e.g., a substantially non-antigenic polymer, such as a polyalkylene oxide or a polyethylene oxide. In certain embodiments, a disclosed glucose oxidase and/or peroxide-degrading enzyme is conjugated to a water soluble polymer, e.g., a hydrophilic polyvinyl polymer, e.g., polyvinylalcohol or polyvinylpyrrolidone. Examples of such polymers include polyalkylene oxide homopolymers such as polyethylene glycol (PEG) or polypropylene glycols, polyoxyethylenated polyols, copolymers thereof and block copolymers thereof. Additional useful polymers include polyoxyalkylenes such as polyoxyethylene, polyoxypropylene, and block copolymers of polyoxyethylene and polyoxypropylene, polymethacrylates, carbomers, and branched or unbranched polysaccharides.

### II. Enzyme Production

Methods for producing glucose oxidase and/or peroxide-degrading enzymes of the invention are known in the art. For example, DNA molecules encoding a glucose oxidase and/or peroxide-degrading enzyme can be chemically synthesized using the sequence information provided herein. Synthetic DNA molecules can be ligated to other appropriate nucleotide sequences, including, e.g., expression control sequences, to produce conventional gene expression constructs encoding the desired glucose oxidase and/or peroxide-degrading enzyme.

Nucleic acids encoding desired glucose oxidase and/or peroxide-degrading enzymes can be incorporated (ligated) into expression vectors, which can be introduced into host cells through conventional transfection or transformation techniques. Transformed host cells can be grown under conditions that permit the host cells to express the genes that encode the glucose oxidase and/or peroxide-degrading enzyme.

In certain embodiments, nucleic acids encoding recombinant glucose oxidase and/or peroxide-degrading enzymes of the invention may be codon optimized for expression in a heterologous cell, e.g., a yeast cell (e.g. a Pichia cell) or an E. coli cell, using methods known in the art.

Specific expression and purification conditions will vary depending upon the expression system employed. For example, if a gene is to be expressed in E. coli, it can be cloned into an expression vector by positioning the engineered gene downstream from a suitable bacterial promoter, e.g., Trp or Tac, and a prokaryotic signal sequence. The expressed secreted protein accumulates in refractile or inclusion bodies, and can be harvested after disruption of the cells by French press or sonication. The refractile bodies then are solubilized, and the proteins refolded and cleaved by methods known in the art.

A glucose oxidase and/or peroxide-degrading enzyme can be produced by growing (culturing) a host cell transfected with an expression vector encoding such glucose oxidase and/or peroxide-degrading enzyme, under conditions that permit expression of the glucose oxidase and/or peroxide-degrading enzyme. Following expression, the glucose oxidase and/or peroxide-degrading enzyme can be harvested and purified or isolated using techniques known in the art, e.g., affinity tags such as glutathione-S-transferase (GST) and histidine tags.

In certain embodiments, a glucose oxidase and/or peroxide-degrading enzyme is dried, e.g., spray-dried. Pharmaceutical proteins may be dried in many ways, e.g., by removal of water, organic solvent or liquid polymer by means including drying with N₂, air or inert gases, vacuum oven drying, lyophilization, washing with a volatile organic solvent followed by evaporation of the solvent, evaporation in a fume hood, tray drying, fluid bed drying, spray drying, vacuum drying, or roller drying.

Spray drying glucose oxidase and/or peroxide-degrading enzymes allows water to be separated from the glucose oxidase and/or peroxide-degrading enzyme preparation, allowing for continuous production of dry solids in powder, granulate, or agglomerate form from liquid feedstocks such as emulsions and pumpable suspensions. Spray drying involves the atomization of a liquid feedstock comprising glucose oxidase and/or peroxide-degrading enzyme into a spray of droplets and contacting the droplets with hot air or gas in a drying chamber. The atomization process may be conducted using a two-fluid atomizer that mixes the liquid feedstock with a drying gas such as compressed air or nitrogen. Operating conditions and dryer design are selected according to the drying characteristics of the glucose oxidase and/or peroxide-degrading enzyme and the desired powder qualities. Exemplary methods for spray drying enzymes are described in United States Patent Application Publication No. 2015/0353913. The glucose oxidase and peroxide-degrading enzyme may ne spray dried separately or combined and spray dried together.

### III. Pharmaceutical Compositions

For therapeutic use, a glucose oxidase and/or peroxide-degrading enzyme described herein preferably is combined with a pharmaceutically acceptable carrier. The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable carrier” as used herein refers to buffers, carriers, and excipients suitable for use in contact with the tissues of human beings and animals without excessive toxicity, irritation, allergic response, or other problem or complication, commensurate with a reasonable benefit/risk ratio. Pharmaceutically acceptable carriers include any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see, e.g., Martin, Remington's Pharmaceutical Sciences, 15th Ed., Mack Publ. Co., Easton, PA [1975]. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

Pharmaceutical compositions containing a naturally occurring or recombinant glucose oxidase and/or peroxide-degrading enzyme disclosed herein can be presented in a dosage unit form and can be prepared by any suitable method. A pharmaceutical composition should be formulated to be compatible with its intended route of administration. The pharmaceutical compositions may be in a variety of forms. These include, for example, liquid, semi-solid and solid dosage forms, such as liquid solutions, dispersions or suspensions, tablets, pills, powders, liposomes and suppositories. The preferred form will depend upon the intended mode of administration and therapeutic application.

Although the compositions preferably are formulated for administration enterally (for example, orally), such compositions can be administered by a parenteral mode (e.g., intravenous, subcutaneous, intraperitoneal, or intramuscular injection). The phrases “parenteral administration” and “administered parenterally” as used herein mean modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and infrasternal injection and infusion.

The composition can be formulated as a solution, microemulsion, dispersion, liposome, or other ordered structure suitable for stable storage at high concentration. Sterile injectable solutions can be prepared by incorporating an agent described herein in the required amount in an appropriate solvent with one or a combination of ingredients enumerated above, as required, followed by filtered sterilization. Generally, dispersions are prepared by incorporating an agent described herein into a sterile vehicle that contains a basic dispersion medium and the required other ingredients from those enumerated above. In the case of sterile powders for the preparation of sterile injectable solutions, the preferred methods of preparation are vacuum drying and freeze drying that yield a powder of an agent described herein plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

Depending upon the mode of administration, for example, by parenteral administration, it may be desirable to produce a pharmaceutical formulation that is sterile. Sterilization can be accomplished by any suitable method, e.g., filtration through sterile filtration membranes. Where the composition is lyophilized, filter sterilization can be conducted prior to or following lyophilization and reconstitution.

In certain embodiments, a disclosed composition comprises a polyionic reagent which may, e.g., coat the glucose oxidase and/or peroxide-degrading enzyme (e.g., the composition comprises a polyionic coating). Exemplary polyionic reagents include PSS (poly (Sodium 4-styrenesulfonate), PAA (poly Acrylic acid sodium salt), PMG (poly (methylene-co-guanidine) hydrochloride), DS (dextran sulfate), PMA (poly (methyl acrylate)), or PVS (polyvinylsiloxane).

In certain embodiments, a disclosed composition and/or dosage comprises: (i) about 750 to about 250,000, about 750 to about 200,000, about 750 to about 150,000, about 750 to about 100,000, 750 to about 75,000, about 750 to about 60,000, about 750 to about 45,000, about 750 to about 30,000, about 750 to about 15,000, about 750 to about 10,000, about 750 to about 7,500, about 750 to about 5,000, about 750 to about 2,500, about 750 to about 1,000, about 1,000 to about 250,000, about 1,000 to about 200,000, about 1,000 to about 150,000, about 1,000 to about 100,000, 1,000 to about 75,000, about 1,000 to about 60,000, about 1,000 to about 45,000, about 1,000 to about 30,000, about 1,000 to about 15,000, about 1,000 to about 10,000, about 1,000 to about 7,500, about 1,000 to about 5,000, about 1,000 to about 2,500, about 2,500 to about 250,000, about 2,500 to about 200,000, about 2,500 to about 150,000, about 2,500 to about 100,000, 2,500 to about 75,000, about 2,500 to about 60,000, about 2,500 to about 45,000, about 2,500 to about 30,000, about 2,500 to about 15,000, about 2,500 to about 10,000, about 2,500 to about 7,500, about 2,500 to about 5,000, about 5,000 to about 250,000, about 5,000 to about 200,000, about 5,000 to about 150,000, about 5,000 to about 100,000, 5,000 to about 75,000, about 5,000 to about 60,000, about 5,000 to about 45,000, about 5,000 to about 30,000, about 5,000 to about 15,000, about 5,000 to about 10,000, about 5,000 to about 7,500, about 7,500 to about 250,000, about 7,500 to about 200,000, about 7,500 to about 150,000, about 7,500 to about 100,000, 7,500 to about 75,000, about 7,500 to about 60,000, about 7,500 to about 45,000, about 7,500 to about 30,000, about 7,500 to about 15,000, about 7,500 to about 10,000, about 10,000 to about 250,000, about 10,000 to about 200,000, about 10,000 to about 150,000, about 10,000 to about 100,000, 10,000 to about 75,000, about 10,000 to about 60,000, about 10,000 to about 45,000, about 10,000 to about 30,000, about 10,000 to about 15,000, about 15,000 to about 250,000, about 15,000 to about 200,000, about 15,000 to about 150,000, about 15,000 to about 100,000, 15,000 to about 75,000, about 15,000 to about 60,000, about 15,000 to about 45,000, about 15,000 to about 30,000, about 30,000 to about 250,000, about 30,000 to about 200,000, about 30,000 to about 150,000, about 30,000 to about 100,000, about 30,000 to about 75,000, about 30,000 to about 60,000, about 30,000 to about 45,000, about 45,000 to about 250,000, about 45,000 to about 200,000, about 45,000 to about 150,000, about 45,000 to about 100,000, about 45,000 to about 75,000, about 45,000 to about 60,000, about 60,000 to about 250,000, about 60,000 to about 200,000, about 60,000 to about 150,000, about 60,000 to about 100,000, about 60,000 to about 75,000, about 75,000 to about 250,000, about 75,000 to about 200,000, about 75,000 to about 150,000, about 75,000 to about 100,000, about 100,000 to about 250,000, about 100,000 to about 200,000, about 100,000 to about 150,000, about 150,000 to about 250,000, about 150,000 to about 200,000, about 200,000 to about 250,000 international units (I.U.) of glucose oxidase enzyme; and/or (ii) about 750 to about 250,000, about 750 to about 200,000, about 750 to about 150,000, about 750 to about 100,000, 750 to about 75,000, about 750 to about 60,000, about 750 to about 45,000, about 750 to about 30,000, about 750 to about 15,000, about 750 to about 10,000, about 750 to about 7,500, about 750 to about 5,000, about 750 to about 2,500, about 750 to about 1,000, about 1,000 to about 250,000, about 1,000 to about 200,000, about 1,000 to about 150,000, about 1,000 to about 100,000, 1,000 to about 75,000, about 1,000 to about 60,000, about 1,000 to about 45,000, about 1,000 to about 30,000, about 1,000 to about 15,000, about 1,000 to about 10,000, about 1,000 to about 7,500, about 1,000 to about 5,000, about 1,000 to about 2,500, about 2,500 to about 250,000, about 2,500 to about 200,000, about 2,500 to about 150,000, about 2,500 to about 100,000, 2,500 to about 75,000, about 2,500 to about 60,000, about 2,500 to about 45,000, about 2,500 to about 30,000, about 2,500 to about 15,000, about 2,500 to about 10,000, about 2,500 to about 7,500, about 2,500 to about 5,000, about 5,000 to about 250,000, about 5,000 to about 200,000, about 5,000 to about 150,000, about 5,000 to about 100,000, 5,000 to about 75,000, about 5,000 to about 60,000, about 5,000 to about 45,000, about 5,000 to about 30,000, about 5,000 to about 15,000, about 5,000 to about 10,000, about 5,000 to about 7,500, about 7,500 to about 250,000, about 7,500 to about 200,000, about 7,500 to about 150,000, about 7,500 to about 100,000, 7,500 to about 75,000, about 7,500 to about 60,000, about 7,500 to about 45,000, about 7,500 to about 30,000, about 7,500 to about 15,000, about 7,500 to about 10,000, about 10,000 to about 250,000, about 10,000 to about 200,000, about 10,000 to about 150,000, about 10,000 to about 100,000, 10,000 to about 75,000, about 10,000 to about 60,000, about 10,000 to about 45,000, about 10,000 to about 30,000, about 10,000 to about 15,000, about 15,000 to about 250,000, about 15,000 to about 200,000, about 15,000 to about 150,000, about 15,000 to about 100,000, 15,000 to about 75,000, about 15,000 to about 60,000, about 15,000 to about 45,000, about 15,000 to about 30,000, about 30,000 to about 250,000, about 30,000 to about 200,000, about 30,000 to about 150,000, about 30,000 to about 100,000, about 30,000 to about 75,000, about 30,000 to about 60,000, about 30,000 to about 45,000, about 45,000 to about 250,000, about 45,000 to about 200,000, about 45,000 to about 150,000, about 45,000 to about 100,000, about 45,000 to about 75,000, about 45,000 to about 60,000, about 60,000 to about 250,000, about 60,000 to about 200,000, about 60,000 to about 150,000, about 60,000 to about 100,000, about 60,000 to about 75,000, about 75,000 to about 250,000, about 75,000 to about 200,000, about 75,000 to about 150,000, about 75,000 to about 100,000, about 100,000 to about 250,000, about 100,000 to about 200,000, about 100,000 to about 150,000, about 150,000 to about 250,000, about 150,000 to about 200,000, about 200,000 to about 250,000 international units (I.U.) of peroxide-degrading enzyme.

Depending upon the circumstances, the ratio of the number of international units of glucose oxidase to the number of international units of the peroxidase-degrading enzyme is in the range of 0.1 to 10, for example, 0.1 to 9, 0.1 to 8, 0.1 to 7, 0.1 to 6, 0.1 to 5, 0.1 to 4, 0.1 to 3, 0.1 to 1 or 0.1 to 1, 0.5 to 10, 0.5 to 9, 0.5 to 8, 0.5 to 7, 0.5 to 6, 0.5 to 5, 0.5 to 4, 0.5 to 3 or 0.5 to 2, 0.5 to 1, 1 to 10, 1 to 9, 1 to 8, 1 to 7, 1 to 6, 1 to 5, 1 to 4, 1 to 3 or 1 to 2.

Depending upon the circumstances, the amount of glucose oxidase and/or the peroxidase degrading example administered to a subject in a particular dosing can range from about 50 to about 20,000, about 50 to about 15,000, about 50 to about 10,000, about 50 to about 7,500, about 50 to about 5,000, about 50 to about 4,000, about 50 to about 3000, about 50 to about 2000, about 50 to about 1,000, about 50 to about 750, about 50 to about 500, or about 50 to about 150 international units/kg, for example, about 20,000, about 15,000, about 10,000, about 5,000, about 4,000, about 3,000, 2,000, 1,000 international units/kg body weight.

For a 180 kg human, it is contemplated that about 500,000 to about 4,000,000, 500,000 to about 3,000,000, about 500,000 to about 2,000,000, 500,000 to about 1,000,000, 1,000,000 to about 4,000,000 1,000,000 to about 3,000,000, or 1,000,000 to about 2,000,000 international units of glucose oxidase and/or peroxidase degrading enzyme may be administered in a single dose.

In certain embodiments, a disclosed glucose oxidase and/or peroxide-degrading enzyme or composition is administered to a subject together with a meal or snack. In certain embodiments, a disclosed glucose oxidase and/or peroxide-degrading enzyme or composition is administered to a subject together with each meal or snack that the subject eats. In certain embodiments, a disclosed glucose oxidase and/or peroxide-degrading enzyme or composition is administered to a subject once every 7 days, once every 6 days, once every 5 days, once every 4 days, once every 3 days, once every 2 days, once every day, 2 times every day, 3 times every day, 4 times every day, 5 times every day, 6 times every, or more than 6 times every day.

Depending upon the circumstances, the composition can be formulated as a powder, granulate, pellet, micropellet, a minitablet, a nanoparticles, or a nanopreparation. The composition can be encapsulated in a capsule, e.g., a hydroxypropyl methylcellulose (HPMC) capsule, soft gelatin capsule, or a hard gelatin capsule. Alternatively, the composition can be formulated as a tablet dosage form. The composition may also be formulated as a liquid oral dosage form, for example, an elixir, syrup, or drop.

In certain embodiments, a disclosed composition does not include (and/or is not administered in combination with) a glucoamylase enzyme. In certain embodiments, a disclosed composition does not include (and/or is not administered in combination with) a sucrase enzyme. In certain embodiments, a disclosed composition does not include (and/or is not administered in combination with) a lactase enzyme. In certain embodiments, a disclosed composition does not include (and/or is not administered in combination with) a transglucosidase enzyme. In certain embodiments, a disclosed composition does not include (and/or is not administered in combination with) an alpha-amylase enzyme. In certain embodiments, a disclosed composition does not include (and/or is not administered in combination with) any of a glucoamylase enzyme, a sucrase enzyme, a lactase enzyme, a transglucosidase enzyme, and/or an alpha-amylase enzyme.

### IV. Therapeutic Uses

The methods and compositions disclosed herein can be used to treat diseases or disorders. For example, the invention provides a method of treating diabetes, obesity, metabolic syndrome, hyperglycemia, hyperinsulinemia, and/or an eating disorder (such as bulimia or carbohydrate hyperphagia) in a subject. The method comprises administering to the subject an effective amount of (i) a glucose oxidase enzyme, and (ii) a peroxide-degrading enzyme. For example, the method may comprise administering a disclosed pharmaceutical composition comprising (i) a spray-dried or lyophilized glucose oxidase enzyme, and (ii) a spray-dried or lyophilized peroxide-degrading enzyme.

The term “effective amount” as used herein refers to the amount of an active agent (e.g., a disclosed glucose oxidase and/or peroxide-degrading enzyme) sufficient to effect beneficial or desired results. An effective amount can be administered in one or more administrations, applications or dosages and is not intended to be limited to a particular formulation or administration route.

As used herein, “treat”, “treating” and “treatment” mean the treatment of a disease in a subject, e.g., in a human. This includes: (a) inhibiting the disease, i.e., arresting its development; and (b) relieving the disease, i.e., causing regression of the disease state. As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably includes humans.

The invention also provides a method of reducing a level of glucose in a subject, for example, a subject with obesity, diabetes, hyperglycemia, and/or an eating disorder (such as bulimia or carbohydrate hyperphagia). The method comprises administering to the subject an effective amount of (i) a glucose oxidase enzyme, and (ii) a peroxide-degrading enzyme. For example, the method may comprise administering a disclosed pharmaceutical composition comprising (i) a spray-dried or lyophilized glucose oxidase, and (ii) a spray-dried or lyophilized peroxide-degrading enzyme. A level of glucose in a subject may refer to a level of glucose in a body fluid (e.g., blood, plasma, serum, or urine), tissue, organ, cell, and/or gastrointestinal tract in the subject, or a sample of any of the foregoing.

Glucose levels, e.g., blood glucose levels, may be measured by any method known in the art, including, for example, glucometers and/or test strips as described in Example 1 herein.

In certain embodiments, the method reduces a level of glucose in a subject (e.g., in the blood of a subject) that is measured 5, 15, 30, 60, 90, 120, 180, 240, 300, or 360 minutes after the subject cats a meal or a snack. In certain embodiments, the method reduces a level of glucose in the subject (e.g., in the blood of a subject) that is measured and/or plotted over a period of time, for example, the method reduces a level of glucose over a period of time determined according to an area under curve (AUC) or Cmax analysis, for example, as described in Example 1 herein. For example, in certain embodiments, the method reduces a level of glucose in the subject from about 0 to about 5 minutes, about 0 to about 15 minutes, about 0 to about 30 minutes, about 0 to about 60 minutes, about 0 to about 90 minutes, about 0 to about 120 minutes, about 0 to about 180 minutes, about 0 to about 240 minutes, about 0 to about 300 minutes, about 0 to about 360 minutes, about 5 to about 15 minutes, about 5 to about 30 minutes, about 5 to about 60 minutes, about 5 to about 90 minutes, about 5 to about 120 minutes, about 5 to about 180 minutes, about 5 to about 240 minutes, about 5 to about 300 minutes, or about 5 to about 360 minutes after the subject cats a meal or a snack.

In certain embodiments, the method reduces a level of glucose in a subject (e.g., in the blood of the subject, e.g., following a meal or snack) by at least about 5%, 10%, 20%, 30%, 40%, 50%, 60%, 70%, 80%, or 90% relative to the same subject when that subject has not been administered a glucose oxidase and a peroxide-degrading enzyme, or relative to a similar subject that has not been administered a glucose oxidase and a peroxide-degrading enzyme. In certain embodiments, the method reduces a level of glucose in a subject (e.g., in the blood of a subject, e.g., following a meal or snack) by from 10% to about 90%, from about 10% to about 80%, from about 10% to about 60%, from about 10% to about 40%, from about 10% to about 20%, from about 20% to about 80%, from about 20% to about 60%, from about 20% to about 40%, from about 40% to about 80%, from about 40% to about 60%, or from about 60% to about 80% relative to the same subject when that subject has not been administered a glucose oxidase and a peroxide-degrading enzyme, or relative to a similar subject that has not been administered a glucose oxidase and a peroxide-degrading enzyme.

In certain embodiments, the method reduces glucose Cmax in the blood of a subject following a meal or snack by at least about 5%, 10%, 20%, 30%, 40%, 50%, 60%, 70%, 80%, or 90% relative to the same subject when that subject has not been administered a glucose oxidase and a peroxide-degrading enzyme, or relative to a similar subject that has not been administered a glucose oxidase and a peroxide-degrading enzyme. In certain embodiments, the method reduces glucose Cmax in the blood of a subject following a meal or snack by from about 10% to about 80%, from about 10% to about 60%, from about 10% to about 40%, from about 10% to about 20%, from about 20% to about 80%, from about 20% to about 60%, from about 20% to about 40%, from about 40% to about 80%, from about 40% to about 60%, or from about 60% to about 80% relative to the same subject when that subject has not been administered a glucose oxidase and a peroxide-degrading enzyme, or relative to a similar subject that has not been administered a glucose oxidase and a peroxide-degrading enzyme.

In certain embodiments, the method reduces glucose Tmax in the blood of a subject following a meal or snack by at least about 5%, 10%, 20%, 30%, 40%, 50%, 60%, 70%, 80%, or 90% relative to the same subject when that subject has not been administered a glucose oxidase and a peroxide-degrading enzyme, or relative to a similar subject that has not been administered a glucose oxidase and a peroxide-degrading enzyme. In certain embodiments, the method reduces glucose Tmax in the blood of a subject following a meal or snack by from about 10% to about 80%, from about 10% to about 60%, from about 10% to about 40%, from about 10% to about 20%, from about 20% to about 80%, from about 20% to about 60%, from about 20% to about 40%, from about 40% to about 80%, from about 40% to about 60%, or from about 60% to about 80% relative to the same subject when that subject has not been administered a glucose oxidase and a peroxide-degrading enzyme, or relative to a similar subject that has not been administered a glucose oxidase and a peroxide-degrading enzyme.

In certain embodiments, when administered together with a food, the method reduces the glycemic index of the food by at least about 5%, 10%, 20%, 30%, 40%, 50%, 60%, 70%, 80%, or 90% relative to the same subject when that subject has not been administered a glucose oxidase and a peroxide-degrading enzyme, or relative to a similar subject that has not been administered a glucose oxidase and a peroxide-degrading enzyme. In certain embodiments, when administered together with a food, the method reduces the glycemic index of the food by at least from about 10% to about 80%, from about 10% to about 60%, from about 10% to about 40%, from about 10% to about 20%, from about 20% to about 80%, from about 20% to about 60%, from about 20% to about 40%, from about 40% to about 80%, from about 40% to about 60%, or from about 60% to about 80% relative to the same subject when that subject has not been administered a glucose oxidase and a peroxide-degrading enzyme, or relative to a similar subject that has not been administered a glucose oxidase and a peroxide-degrading enzyme.

In certain embodiments, when administered together with a food, the method reduces the caloric intake of the food by at least about 5%, 10%, 20%, 30%, 40%, 50%, 60%, 70%, 80%, or 90% relative to the same subject when that subject has not been administered a glucose oxidase and a peroxide-degrading enzyme, or relative to a similar subject that has not been administered a glucose oxidase and a peroxide-degrading enzyme. In certain embodiments, when administered together with a food, the method reduces the caloric intake of the food by at least from about 10% to about 80%, from about 10% to about 60%, from about 10% to about 40%, from about 10% to about 20%, from about 20% to about 80%, from about 20% to about 60%, from about 20% to about 40%, from about 40% to about 80%, from about 40% to about 60%, or from about 60% to about 80% relative to the same subject when that subject has not been administered a glucose oxidase and a peroxide-degrading enzyme, or relative to a similar subject that has not been administered a glucose oxidase and a peroxide-degrading enzyme.

The invention also provides a method of reducing a level of C-peptide and/or insulin in a subject, for example, a subject with obesity, diabetes, hyperglycemia, and/or an eating disorder (such as bulimia or carbohydrate hyperphagia). The method comprises administering to the subject an effective amount of (i) a glucose oxidase enzyme, and (ii) a peroxide-degrading enzyme. For example, the method may comprise administering a disclosed pharmaceutical composition comprising (i) a spray-dried or lyophilized glucose oxidase, and (ii) a spray-dried or lyophilized peroxide-degrading enzyme. A level of C-peptide and/or insulin in a subject may refer to a level of C-peptide and/or insulin in a body fluid (e.g., blood, plasma, serum, or urine), tissue, organ, cell, and/or gastrointestinal tract in the subject, or a sample of any of the foregoing. C-peptide and/or insulin levels, e.g., blood C-peptide levels, may be measured by any method known in the art, including, for example, by ELISA as described in Example 1 herein.

In certain embodiments, the method reduces a level of C-peptide and/or insulin in a subject (e.g., in the blood of a subject) that is measured 5, 15, 30, 60, 90, 120, 180, 240, 300, or 360 minutes after the subject cats a meal or a snack. In certain embodiments, the method reduces a level of C-peptide and/or insulin in the subject (e.g., in the blood of the subject) that is measured and/or plotted over a period of time, for example, the method reduces a level of glucose over a period of time determined according to an area under curve (AUC) or Cmax analysis, for example, as described in Example I herein. For example, in certain embodiments, the method reduces a level of C-peptide and/or insulin in the subject that is measured from about 0 to about 5 minutes, about 0 to about 15 minutes, about 0 to about 30 minutes, about 0 to about 60 minutes, about 0 to about 90 minutes, about 0 to about 120 minutes, about 0 to about 180 minutes, about 0 to about 240 minutes, about 0 to about 300 minutes, about 0 to about 360 minutes, about 5 to about 15 minutes, about 5 to about 30 minutes, about 5 to about 60 minutes, about 5 to about 90 minutes, about 5 to about 120 minutes, about 5 to about 180 minutes, about 5 to about 240 minutes, about 5 to about 300 minutes, or about 5 to about 360 minutes after the subject cats a meal or a snack.

In certain embodiments, the method reduces a level of C-peptide and/or insulin in a subject (e.g., in the blood of the subject, e.g., following a meal or snack) by at least about 5%, 10%, 20%, 30%, 40%, 50%, or 60% relative to the same subject when that subject has not been administered a glucose oxidase and a peroxide-degrading enzyme, or relative to a similar subject that has not been administered a glucose oxidase and a peroxide-degrading enzyme. In certain embodiments, the method reduces a level of C-peptide and/or insulin in a subject (e.g., in the blood of a subject, e.g., following a meal or snack) by from about 5% to about 60%, from about 5% to about 40%, from about 5% to about 20%, from about 5% to about 10%, from about 10% to about 60%, from about 10% to about 40%, from about 10% to about 20%, from about 20% to about 60%, from about 20% to about 40%, or from about 40% to about 60% relative to the same subject when that subject has not been administered a glucose oxidase and a peroxide-degrading enzyme, or relative to a similar subject that has not been administered a glucose oxidase and a peroxide-degrading enzyme.

The invention also provides a method of reducing a level of HAlc in a subject, for example, a subject with obesity, diabetes, hyperglycemia, and/or an eating disorder (such as bulimia or carbohydrate hyperphagia). The method comprises administering to the subject an effective amount of (i) a glucose oxidase enzyme, and (ii) a peroxide-degrading enzyme. For example, the method may comprise administering a disclosed pharmaceutical composition comprising (i) a spray-dried or lyophilized glucose oxidase, and (ii) a spray-dried or lyophilized peroxide-degrading enzyme. A level of HAlc in a subject may refer to a level of HAlc in a body fluid (e.g., blood, plasma, serum, or urine), tissue, organ, cell, and/or gastrointestinal tract in the subject, or a sample of any of the foregoing. HAlc levels may be measured by any method known in the art.

The methods and compositions described herein can be used alone or in combination with other therapeutic agents and/or modalities. The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### EXAMPLES

The following Examples are merely illustrative and are not intended to limit the scope or content of the invention in any way.

### Example 1

This Example describes a study testing the effect of a combination of glucose oxidase and catalase, homogenously mixed with dry feed, on glucose absorption from the intestinal lumen and C-peptide release in young, healthy pigs.

### Study Design/Methods

A porcine model was used due to similarities between humans and pigs in morphology and physiology of gastrointestinal systems, enzymatic and hormonal factors, transit times, and digestive efficiencies. Both healthy and diabetic pigs have been used to test glucose tolerance and insulin sensitivity (see, for example, Koopmans et al. (2011) BMC BIOCHEM 12:25, and Pierzynowska et al. (2020) J. DIABETES RES. 2020:2148740).

The study enrolled eight pigs (4 females and 4 castrated males). All pigs were 10-12 weeks old upon arrival at the experimental site, and had a weight of 9.06±0.77 kg (mean±SD) at the start of the study.

For a one week adaptation period, pigs were fed with standard cereal-based feed (SD, Kcynia, Morawski Plant, Poland). To ensure higher and consistent stomach pH levels standard feed (0.5% of body weight) was offered to pigs in 3 hour intervals starting with a morning feeding at 0900 hours (8 times for 24 hours; 0900, 1200, 1500, 1800, 2100, 2400, 0300, and 0600 hours). The composition of the standard feed is provided in TABLE 1. The standard feed includes approximately 50% starch, 17% protein, 3% fat, 28% fiber, and 2% H2O. As the standard feed contains approximately 50% starch, 4 grams of feed contains approximately 2 grams of starch, corresponding to 2 grams of glucose.

TABLE 1
Standard feed composition

| Raw Material | Amount (grams) |
|---|---|
| Wheat | 40.275 |
| Barley | 40.000 |
| Soya meal | 12.000 |
| Fish Flavour | 3.000 |
| Monocalcium Phosphorum | 2.000 |
| Fodder Chalk | 1.000 |
| Fodder Salt | 0.270 |
| L-Lysine 78% | 0.500 |
| DL-Methionine 99% | 0.270 |
| L-Treonine 98,5% | 0.20 |
| L-Tryptophan 98% | 0.200 |
| Organic Acids | 0.13 |
| Zinc Oxide | 0.042 |
| Vitamin Premix | 0.013 |
| Micronutrients | 0.100 |
| Total | 100.000 |

One week after arrival, at the end of adaptation period, the pigs had jugular catheters implanted to allow for frequent blood sampling throughout the study.

After the initial adaptation period and jugular catheter insertion, pigs were subjected to Meal Glucose Tolerance Tests (MGTTs; see TABLE 3) where feed or glucose-enriched feed was either supplemented with glucose oxidase and catalase enzymes or not. The effect of enzyme supplementation on glucose utilization and C-peptide release was monitored.

Details of the glucose oxidase and catalase enzymes used in the study are provided in TABLE 2

TABLE 2
Glucose Oxidase and Catalase Enzymes

| Enzyme Name | Glucose Oxidase | Glucose Oxidase | Catalase |
|---|---|---|---|
| Organism | Aspergillus niger | Aspergillus niger | Aspergillus niger |
| Source, Cat. | Sigma, G7141 | Sigma, G7141 | Sigma, C3515 |
| number | | | |
| Lot # | SLCB1875 | SLCGO0619 | SLCF3828 |
| Specific activity | 145200 units/g | 167978 units/g | 9299 units/mg protein, |
| | | | 10.7 mg protein/ml |
| | | | suspension |
| Formulation | lyophilized | lyophilized | suspension in |
| | powder | powder | ammonium sulfate |
| Storage and | −20° C. | −20° C. | 4° C. |
| handling | | | |
| Experiments used | MGTT IV, V | MGTT VI | MGTT IV, V, VI |
| Dose per | MGTT IV - 65,340 | 75,590.1 | MGTT IV - 79,599.44 |
| pig (units) | MGTT V - 130,680 | | MGTT V - 159,198.88 |
| | | | MGTT VI - 79,599.44 |

Blood samples were collected via the jugular vein catheter and transferred to BD Vacutainer® glass K₃EDTA tubes (BD Diagnostics, New Jersey, USA). The blood samples were immediately placed on ice before they were centrifuged at 3000×g for 15 minutes at 4° C., and plasma was separated and stored at −80° C. until further analysis.

Blood glucose concentrations were measured directly following blood sampling using glucometers and test strips (Accu-Chek Aviva, Roche Diagnostics, Germany). Plasma C-peptide concentrations were measured using porcine C-peptide ELISA kits (cat #10-1256-01 Mercodia, Uppsala, Sweden), according to the manufacturer's instructions.

A summary of the study design and the individual Meal Glucose Tolerance Test (MGTT) conditions are described in TABLE 3. All MGTTs were performed in the morning, beginning at 0900 hours. On each test day blood was sampled before (base line) and at 5, 15, 30, 60, 90, 120, 180, and 240 minutes after feeding. Optionally, blood was additionally sampled at 300 and 360 minutes after feeding. During test days meals at 0600 hours and 1200 hours were not given to pigs.

Initially a set of MGTTs (MGTTs I, II, and III) were performed to find an optimal amount of feed and feed/glucose ratio. MGTT I and II revealed that feeding pigs with 2 grams glucose per kilogram body weight did not create a sufficient peak. In MGTT III pigs were fed (i) 16 grams standard feed per kilogram body weight, or (ii) 16 grams standard feed per kilogram body weight and 8 grams glucose per kilogram body weight (mixed with feed). In MGTT III, a glucose absorption peak was observed both for feed and feed/glucose mixture. As a result, this amount was used in all further MGTTs.

Further MGTTs (MGTTs IV, V, and VI) tested the effect of glucose oxidase and catalase supplementation. Glucose oxidase powder was added and homogenized with feed or feed/glucose mixture. Catalase suspension was added in drops from a pipette directly to the feed/glucose oxidase or feed/glucose oxidase/glucose mixtures. This process ensured homogenization of the individual components.

At the end of the experiment, pigs were euthanized with sodium pentobarbiturate (Morbital, Biowet, Pulawy, Poland; 20 mg/kg) and submitted to gross post-mortem examination. The stomach, as well as proximal and distal jejunum were examined for pathological changes.

TABLE 3
Study design

| Day(s) | Activity |
|---|---|
| 1-6 | Pig transportation and adaptation |
| 7-10 | Surgery (jugular catheter insertion) and postoperative recovery |
| 11 | MGTT I |
| | Group 1: 4 g standard feed/kg body weight (n = 4) |
| | Group 2: 4 g standard feed + 2 g glucose powder/kg body weight (n = 4) |
| | Blood collection up to 240 minutes |
| 13 | MGTT II |
| | Group 1: 12 g standard feed/kg body weight (n = 4) |
| | Group 2: 12 g standard feed + 6 g glucose powder/kg body weight (n = 4) |
| | Blood collection up to 240 minutes |
| 15 | MGTT III |
| | Group 1: 16 g standard feed/kg body weight (n = 4) |
| | Group 2: 16 g standard feed + 8 g glucose powder/kg body weight (n = 4) |
| | Blood collection up to 240 minutes |
| 17 | MGTT IV |
| | Group 1: 16 g standard feed/kg body weight + 450 mg (65,340 U) glucose oxidase + |
| | 0.8 ml catalase (79,599.44 U) (n = 4) |
| | Group 2: 16 g standard feed + 8 g glucose powder/kg body weight + 450 mg |
| | (65,349 U) glucose oxidase + 0.8 ml (79,599.44 U) catalase (n = 4) |
| | Blood collection up to 240 minutes |
| 19 | MGTT V |
| | Group 1 (Control): 16 g standard feed/kg body weight (n = 3) |
| | Group 2 (Treatment): 16 g standard feed/kg body weight + 900 mg (130,680 U) |
| | glucose oxidase + 1.6 ml (159,198.88 U) catalase (n = 4) |
| | Blood collection up to 360 minutes |
| 20 | MGTT VI |
| | Group 1 (Control Feed): 16 g standard feed/kg body weight (n = 3) |
| | Group 2 (Treatment Feed + Glucose): 16 g standard feed + 8 g glucose powder/kg |
| | body weight + 450 mg glucose oxidase (75,590.1 U) + 0.8 ml (79,599.44 U) |
| | catalase (n = 2) |
| | Group 3 (Control Feed+Glucose): 16 g standard feed + 8 g glucose powder/kg body |
| | weight (n = 2) |
| | Blood collection up to 360 minutes |
| 22 | Autopsy |

All data, with the exception of AUCs was expressed as mean±standard deviation (SD). The distribution of the parameters was checked using a Shapiro-Wilk normality test. The total area under the curve (AUC) was calculated for post-prandial blood glucose and C-peptide levels. AUCs were base line adjusted and compared using one-way ANOVA. Data for AUCs was presented as mean±standard error of mean (SEM). Blood glucose and C-peptide levels at different time points were compared using a two-way ANOVA. Data was not corrected for multiple comparisons. In all statistical analyses p≤0.05 was considered significant. All analyses were carried out using Prism, version 9.1.0 (GraphPad Software, Inc, San Diego, CA, USA).

### Results—Glucose Absorption

The study assessed whether glucose oxidase and catalase could reduce absorption of glucose to the blood during a meal glucose tolerance test (MGTT).

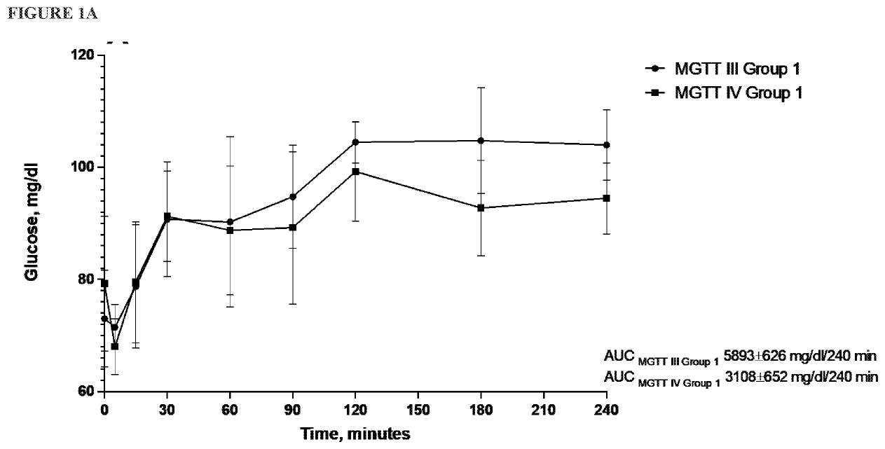

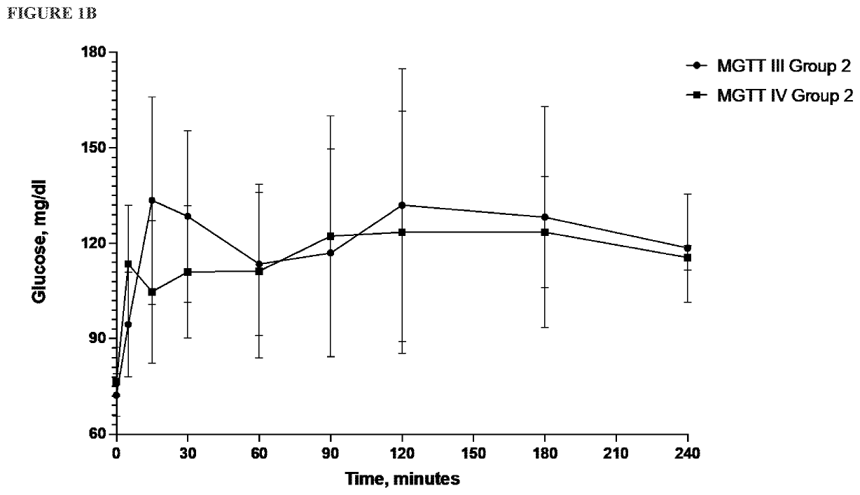

All pigs first received the glucose oxidase and catalase combination during MGTT IV. Results are shown in FIG. 1. Enzyme supplementation did not affect the palatability of feed or pig feeding behaviour. Glucose oxidase and catalase supplementation resulted in a significant decrease in blood glucose levels both in pigs eating feed only (at 180 and 240 minutes after feeding, FIG. 1A) and in pigs eating feed/glucose mixture (at 15 and 30 minutes after feeding, FIG. 1B). A significant (p=0.02) 53% decline in glucose AUC was observed in pigs eating feed only (FIG. 1A).

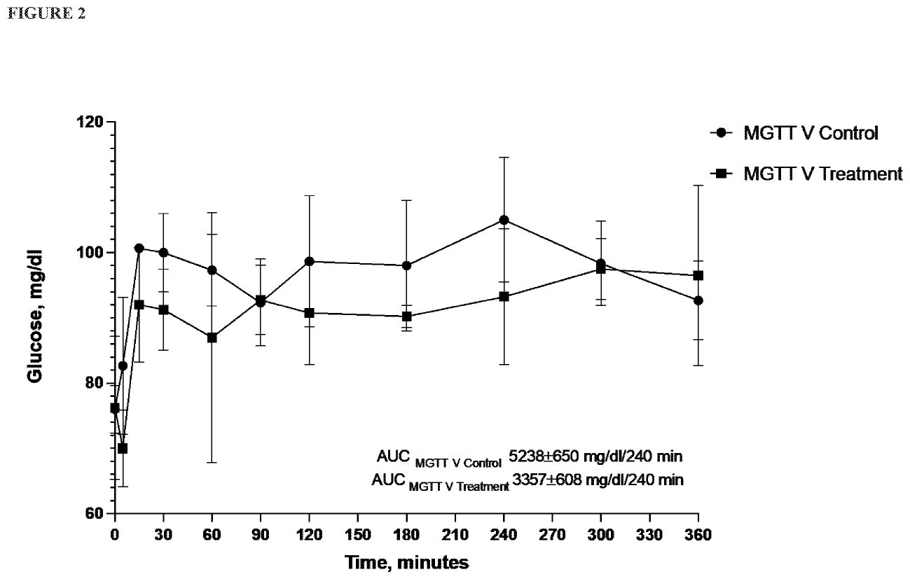

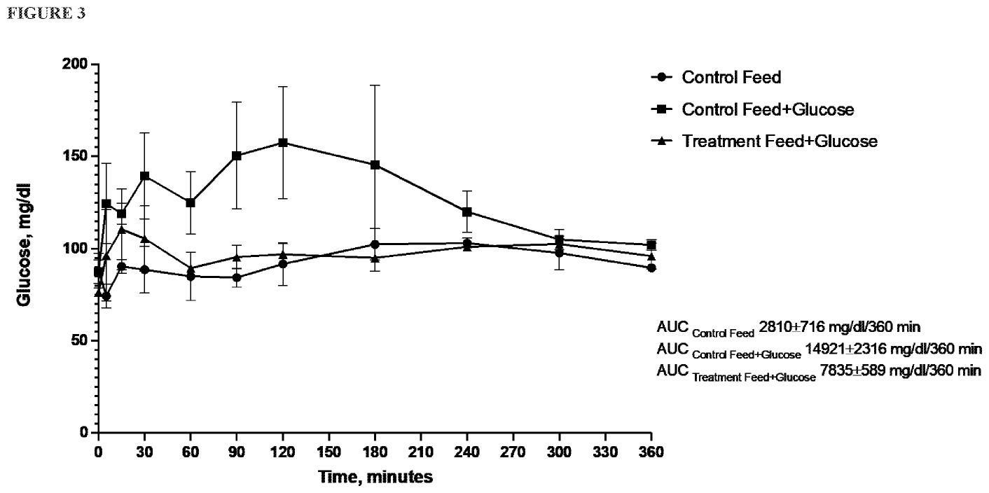

During MGTT V, four pigs received an increased dose of the glucose oxidase and catalase combination relative to MGTT IV. Results are shown in FIG. 2. Results were generally consistent with those for the lower dose. During MGTT VI, three pigs received a meal of feed only, two pigs received a meal of feed/glucose mixture, and two pigs received a meal of feed/glucose mixture as well as the glucose oxidase and catalase combination (at the same dose as in MGTT IV). Results are shown in FIG. 3. A significant 52.5% decrease in glucose AUC was observed in pigs receiving enzyme treatment (p<0.05). A trend difference was observed for glucose Cmax (which was 157.5 mg/dl at 120 minutes for animals receiving the feed/glucose mixture without enzymes, and 110.5 mg/dl at 15 minutes for animals receiving the feed/glucose mixture with enzymes, p=0.06).

### Results—C-Peptide Release

The study also assessed whether glucose oxidase and catalase could reduce release of C-peptide (a clinical marker of insulin) during a meal glucose tolerance test (MGTT).

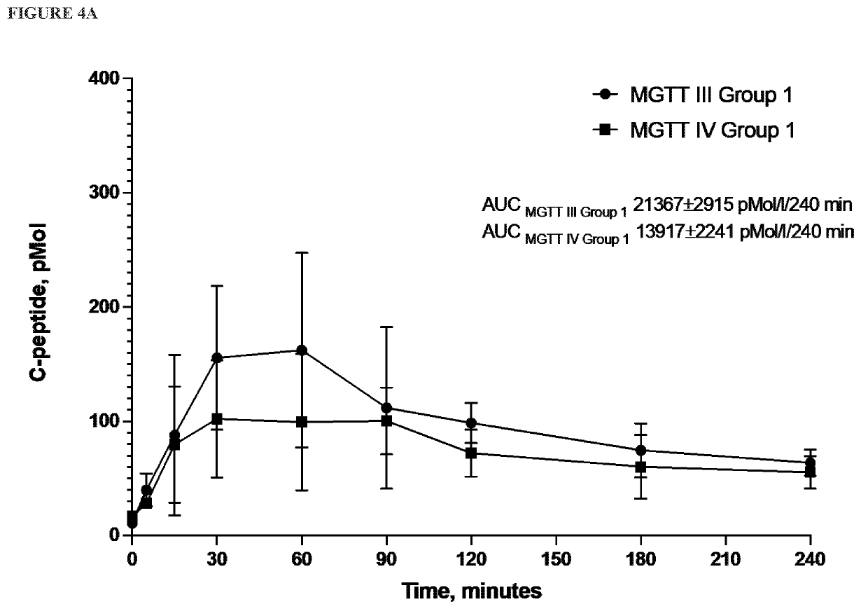

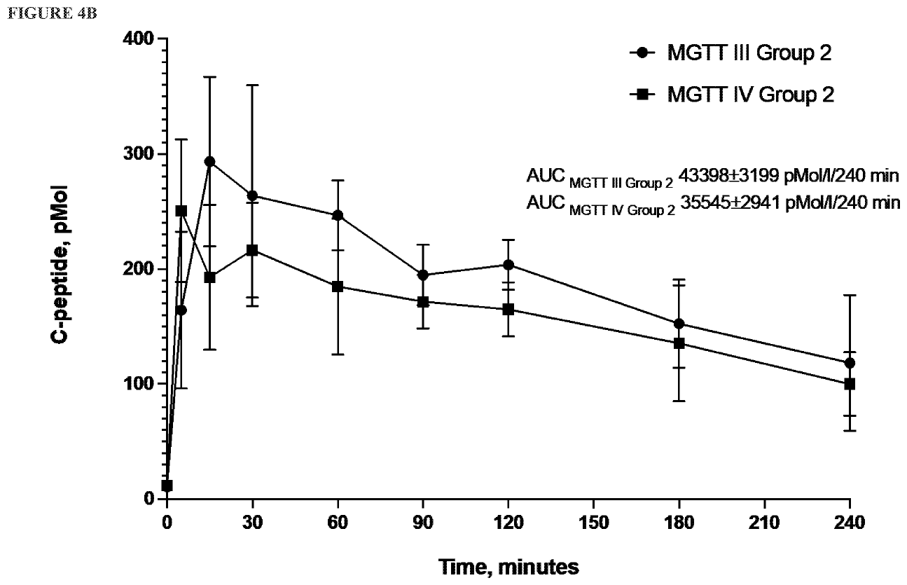

All pigs first received the glucose oxidase and catalase combination during MGTT IV. Results are shown in FIG. 4. Glucose oxidase and catalase supplementation resulted in a trend decrease in C-peptide AUC both for pigs eating feed only FIG. 4A) and in pigs eating feed/glucose mixture (FIG. 4B).

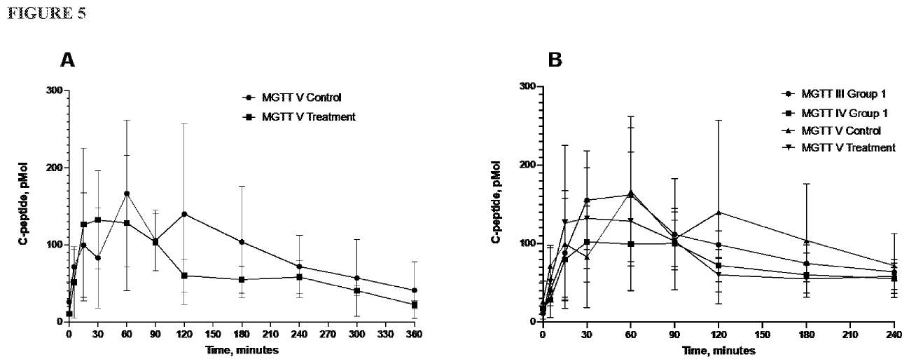

Results for MGTT V are shown in FIG. 5. During MGTT V, there was a trend decrease in C-peptide release curves or AUCs between the control group receiving no enzyme treatment and the treatment group receiving enzyme treatment.

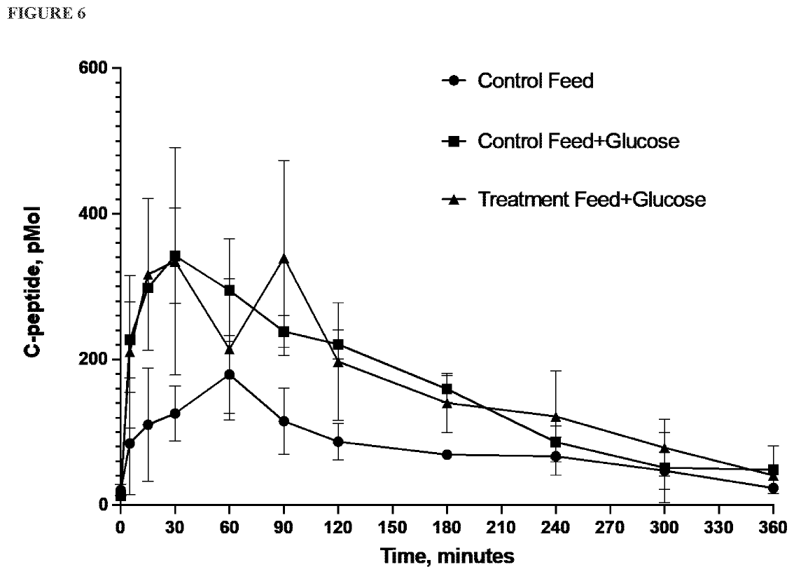

Results for MGTT VI are shown in FIG. 6. During MGTT VI, there was a trend decrease in C-peptide release curves or AUCs between the control group receiving no enzyme treatment and the treatment group receiving enzyme treatment.

### Summary

Throughout the study, no mortality, no adverse clinical signs, and no pathologic macroscopic findings along the gut were observed following the administration of glucose oxidase and catalase enzymes.

Depending upon the experimental conditions, supplementation of feed with glucose oxidase and catalase enzymes resulted in (i) a significant 52% decrease in blood glucose AUC (p<0.05); (ii) a decrease in blood glucose Cmax (30% decrease, p=0.06); and (iii) a decrease in C-peptide AUC (35% decrease for feed and 18% decrease for feed/glucose mixture, p=0.089 and 0.12, respectively).

Together, these results show that feed supplementation with glucose oxidase and catalase enzymes can result in the reduced glucose absorption, and suggest that administration of glucose oxidase and catalase enzymes may treat or prevent obesity or metabolic disorders, such as metabolic syndrome and type 2 diabetes mellitus.

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

SEQUENCE LISTING

SEQ ID NO: 1 (Aspergillus niger glucose oxidase; Uniprot P13006):
MQTLLVSSLVVSLAAALPHYIRSNGIEASLLTDPKDVSGRTVDYIIAGGGLIGLTTAARLTENP
NISVLVIESGSYESDRGPIIEDLNAYGDIFGSSVDHAYETVELATNNQTALIRSGNGLGGSTLV
NGGTWTRPHKAQVDSWETVFGNEGWNWDNVAAYSLQAERARAPNAKQIAAGHYFNASCHGVNGT
VHAGPRDTGDDYSPIVKALMSAVEDRGVPTKKDFGCGDPHGVSMFPNTLHEDQVRSDAAREWLL
PNYQRPNLQVLTGQYVGKVLLSQNGTTPRAVGVEFGTHKGNTHNVYAKHEVLLAAGSAVSPTIL
EYSGIGMKSILEPLGIDTVVDLPVGLNLQDQTTATVRSRITSAGAGQGQAAWFATFNETFGDYS
EKAHELLNTKLEQWAEEAVARGGFHNTTALLIQYENYRDWIVNHNVAYSELFLDTAGVASFDVW
DLLPFTRGYVHILDKDPYLHHFAYDPQYFLNELDLLGQAAATQLARNISNSGAMQTYFAGETIP
GDNLAYDADLSAWTEYIPYHFRPNYHGVGTCSMMPKEMGGVVDNAARVYGVQGLRVIDGSIPPT
QMSSHVMTVFYAMALKISDAILEDYASMQ
SEQ ID NO: 2 (Aspergillus niger glucose oxidase; Uniprot P13006, w/o signal
sequence):
SNGIEASLLTDPKDVSGRTVDYIIAGGGLTGLTTAARLTENPNISVLVIESGSYESDRGPIIED
LNAYGDIFGSSVDHAYETVELATNNQTALIRSGNGLGGSTLVNGGTWTRPHKAQVDSWETVFGN
EGWNWDNVAAYSLQAERARAPNAKQIAAGHYFNASCHGVNGTVHAGPRDTGDDYSPIVKALMSA
VEDRGVPTKKDFGCGDPHGVSMFPNTLHEDQVRSDAAREWLLPNYQRPNLQVLTGQYVGKVLLS
QNGTTPRAVGVEFGTHKGNTHNVYAKHEVLLAAGSAVSPTILEYSGIGMKSILEPLGIDTVVDL
PVGLNLQDQTTATVRSRITSAGAGQGQAAWFATFNETFGDYSEKAHELLNTKLEQWAEEAVARG
GFHNTTALLIQYENYRDWIVNHNVAYSELFLDTAGVASFDVWDLLPFTRGYVHILDKDPYLHHF
AYDPQYFLNELDLLGQAAATQLARNISNSGAMQTYFAGETIPGDNLAYDADLSAWTEYIPYHFR
PNYHGVGTCSMMPKEMGGVVDNAARVYGVQGLRVIDGSIPPTQMSSHVMTVFYAMALKISDAIL
EDYASMQ
SEQ ID NO: 3 (Aspergillus niger catalase; Uniprot A0A254TZH3):
MRHFWLLPAVAGIAGAQCPYLSGEMSFTQEQDNAGDTIEVTEQPIDNTLYVNDTGSYMTTDFGT
PISDQTSLKAGPRGPTLLEDFIFRQKLQRFDHERVPERVVHARGAGAYGTFKSYADWSNVTAAD
FLSANDKETPMFCRFSTVVGFRGSVDTARDVHGHACRFYTDEGNYDIVGINFAPFFIQDAIQFP
DLVHAIKPMPNNEIPQAATAHTSAWDFFSQQSTALHSALWLMSGNGIPRSFRHMNGYGVHSFRF
VAANGTSKVVRYRWKSQQGVASLVWDEAQAAAGKNSDYHRQDLYNAIANGHYPKYELQAQIMDE
ADMLRFGFDLLDPTKLVPEEVVPYTPLGMMELNANPTNYFAEVEQAGFQPGHVVPGIDFTDDPL
LQGRLFSYLDTQLTRHGGPNFEQIPVNRPRKPVHNNNRDGFGQQQIPTNNWAYTPNSMSNGYPM
QANQTQGHGFFTAPYRYASGHLVRQTSPTFNDHWSQPAMFWNSLIPAEQQMVVNAIVFENSKVN
SPHVRKNVVNQLNMVNNNLAVRVARGLGLDEPSPNPTYYTSNKTSNVGTFGKPLLSIEGLQVGF
LASNSHPESIKQGQAMAAQFSAAGVDLNIVTEAYADGVNTTYALSDAIDEDALIIADGVQSLFA
SPALANQMNSTATSTLYPPARPFQILVDSFRYGKPVAAVGSGSVALKNAGIDSSRSGVYTGSSE
TTEKIAKEVLEGLYTFRFVDRFALDE
SEQ ID NO: 4 (Aspergillus niger catalase; Uniprot A0A254TZH3; w/o signal
sequence):
QCPYLSGEMSFTQEQDNAGDTIEVTEQPIDNTLYVNDTGSYMTTDFGTPISDQTSLKAGPRGPT
LLEDFIFRQKLQRFDHERVPERVVHARGAGAYGTFKSYADWSNVTAADFLSANDKETPMFCRFS
TVVGFRGSVDTARDVHGHACRFYTDEGNYDIVGINFAPFFIQDAIQFPDLVHAIKPMPNNEIPQ
AATAHTSAWDFFSQQSTALHSALWLMSGNGIPRSFRHMNGYGVHSFRFVAANGTSKVVRYRWKS
QQGVASLVWDEAQAAAGKNSDYHRQDLYNAIANGHYPKYELQAQIMDEADMLRFGFDLLDPTKL
VPEEVVPYTPLGMMELNANPTNYFAEVEQAGFQPGHVVPGIDFTDDPLLQGRLFSYLDTQLTRH
GGPNFEQIPVNRPRKPVHNNNRDGFGQQQIPTNNWAYTPNSMSNGYPMQANQTQGHGFFTAPYR
YASGHLVRQTSPTFNDHWSQPAMFWNSLIPAEQQMVVNAIVFENSKVNSPHVRKNVVNQLNMVN
NNLAVRVARGLGLDEPSPNPTYYTSNKTSNVGTFGKPLLSIEGLQVGFLASNSHPESIKQGQAM
AAQFSAAGVDLNIVTEAYADGVNTTYALSDAIDFDALIIADGVQSLFASPALANQMNSTATSTL
YPPARPFQILVDSFRYGKPVAAVGSGSVALKNAGIDSSRSGVYTGSSETTEKIAKEVLEGLYTF
RFVDRFALDE

What is claimed is:

1. A pharmaceutical composition comprising a glucose oxidase enzyme, a peroxide-degrading enzyme, and a pharmaceutically acceptable excipient.

2. The pharmaceutical composition of claim 1, wherein the glucose oxidase enzyme is spray-dried or lyophilized.

3. The pharmaceutical composition of claim 1 or 2, wherein the glucose oxidase enzyme is a microbial glucose oxidase enzyme, or a functional fragment or variant thereof.

4. The pharmaceutical composition of any one of claims 1-3, wherein the glucose oxidase enzyme is derived from Aspergillus niger.

5. The pharmaceutical composition of claim 4, wherein the glucose oxidase enzyme comprises SEQ ID NO: 2, or a functional fragment or variant thereof.

6. The pharmaceutical composition of any one of claims 1-5, wherein the composition comprises from about 1,000 to about 250,000 international units (I.U.) of the glucose oxidase enzyme.

7. The pharmaceutical composition of any one of claims 1-6, wherein the peroxide-degrading enzyme is spray-dried or lyophilized.

8. The pharmaceutical composition of any one of claims 1-7, wherein the peroxide-degrading enzyme is a catalase enzyme or a peroxidase enzyme.

9. The pharmaceutical composition of claim 8, wherein the peroxide-degrading enzyme is a catalase enzyme.

10. The pharmaceutical composition of claim 9, wherein the catalase enzyme is a microbial catalase enzyme, or a functional fragment or variant thereof.

11. The pharmaceutical composition of claim 9 or 10, wherein the catalase enzyme is derived from Aspergillus niger.

12. The pharmaceutical composition of claim 11, wherein the catalase enzyme comprises SEQ ID NO: 4, or a functional fragment or variant thereof.

13. The pharmaceutical composition of any one of claims 1-12, wherein the composition comprises from about 1,000 to about 250,000 international units (I.U.) of the peroxide-degrading enzyme.

14. The composition of any one of claims 1-13, wherein the ratio of international units of glucose oxidase to international units of the peroxide-degrading enzyme is in the range from 0.1 to 10.

15. The pharmaceutical composition of any one of claims 1-14, wherein the composition is formulated as an oral dosage form (e.g., a solid or liquid oral dosage form).

16. The pharmaceutical composition of claim 15, wherein the composition is a formulated as a powder, satchel, granulate, pellet, micropellet, tablet, minitablet, elixir, syrup, drop, or nanopreparation.

17. The pharmaceutical composition of claim 16, wherein the composition is formulated as a powder, satchel, or tablet.

18. The pharmaceutical composition of any one of claims 1-17, wherein the composition has a shelf-life at room temperature of at least 3 months, 6 months, 9 months, 12 months, 15 months, 18 months, 21 months, 24 months, 36 months, 48 months, 60 months, 72 months, 84 months, 96 months, 108 months, or 120 months.

19. The pharmaceutical composition of any one of claims 1-18 wherein the composition does not include a glucoamylase enzyme, a sucrase enzyme, a lactase enzyme, a transglucosidase enzyme, an α-amylase enzyme, or a combination thereof.

20. A method of treating diabetes in a subject in need thereof, the method comprising orally administering to the subject the pharmaceutical composition of any one of claims 1-19.

21. The method of claim 20, wherein the diabetes is type 2 diabetes.

22. A method of treating obesity in a subject in need thereof, the method comprising orally administering to the subject the pharmaceutical composition of any one of claims 1-19.

23. A method of treating metabolic syndrome in a subject in need thereof, the method comprising orally administering to the subject the pharmaceutical composition of any one of claims 1-19.

24. A method of treating hyperglycemia in a subject in need thereof, the method comprising orally administering to the subject the pharmaceutical composition of any one of claims 1-19.

25. A method of treating hyperinsulinemia in a subject in need thereof, the method comprising orally administering to the subject the pharmaceutical composition of any one of claims 1-19.

26. A method of reducing a glucose level in a subject, the method comprising orally administering to the subject the pharmaceutical composition of any one of claims 1-19.

27. The method of claim 26, wherein the method reduces the level of glucose in the blood of the subject and/or in the gastrointestinal tract of the subject.

28. The method of claim 26 or 27, wherein the method reduces the level of glucose in a sample from the subject.

29. The method of any one of claims 26-28, wherein the method reduces the level of glucose in the blood of the subject from 0 to 240 minutes or from 0 to 360 minutes following a meal or snack, as determined by an Area Under Curve (AUC) analysis, by at least about 10%, 20%, 30%, 40%, 50%, 60%, 70%, 80%, or 90%.

30. The method of any one of claims 26-29, wherein the method reduces glucose Cmax in the blood of the subject following a meal or snack by at least about 10%, 20%, 30%, 40%, 50%, 60%, 70%, 80%, or 90%.

31. The method of any one of claims 26-30, wherein the method reduces glucose Tmax in the blood of the subject following a meal or snack by at least about 10%, 20%, 30%, 40%, 50%, 60%, 70%, 80%, or 90%.

32. The method of any one of claims 26-31, wherein, when administered together with a food, the method reduces the glycemic load of the food by at least about 10%, 20%, 30%, 40%, 50%, 60%, 70%, 80%, or 90%.

33. The method of any one of claims 26-32, wherein, when administered together with a food, the method reduces the caloric intake of the food by at least about 10%, 20%, 30%, 40%, 50%, 60%, 70%, 80%, or 90%.

34. A method of reducing a C-peptide and/or insulin level in a subject, the method comprising orally administering to the subject the pharmaceutical composition of any one of claims 1-19.

35. The method of claim 34, wherein the method reduces the level of C-peptide and/or insulin in the blood of the subject.

36. The method of claim 34 or 35, wherein the method reduces the level C-peptide and/or insulin in a sample from the subject.

37. The method of any one of claims 34-36, wherein the method reduces the level of C-peptide and/or insulin in the blood of the subject from 0 to 240 minutes or from 0 to 360 minutes following a meal or snack, as determined by an Area Under Curve (AUC) analysis, by at least about 10%, 20%, 30%, 40%, 50%, or 60%.

38. A method of reducing a hemoglobin Alc (HAlc) level in a subject, the method comprising orally administering to the subject the pharmaceutical composition of any one of claims 1-19.

39. The method of claim 38, wherein the method reduces the level of HAlc in the blood of the subject.

40. The method of claim 38 or 39, wherein the method reduces the level of HAlc in a sample from the subject.

41. The method of any one of claims 38-40, wherein the sample is a body fluid sample.

42. The method of claim 41, wherein the fluid sample is blood, serum or plasma.

43. A method of reducing the glycemic load of consumed carbohydrates in carbohydrate-rich diet, the method comprising orally administering to the subject the pharmaceutical composition of any one of claims 1-19.

44. The method of claim 43, wherein administration of the pharmaceutical composition reduces the level of glucose in the gastrointestinal tract of the subject available for absorption.

45. The method of any one of claims 26-44, wherein the subject has diabetes, obesity, metabolic syndrome, hyperglycemia, hyperinsulinemia, and/or an eating disorder.

46. The method of any one of claims 20-45, wherein the pharmaceutical composition is administered to the subject 1, 2, 3, 4, or more than 4 times per day.

47. The method of any one of claims 20-46, wherein the pharmaceutical composition is administered to the subject together with a meal or snack.

48. The method of any one of claims 20-47, wherein the pharmaceutical composition is administered to the subject at each meal or snack.

49. The method of any one of claims 20-48, wherein the subject as a human adult or a human pediatric subject.


SEQUENCE LISTING (4 sequences)

SEQ ID NO: 1 - protein, 605 aa, synthetic construct
MQTLLVSSLVVSLAAALPHYIRSNGIEASLLTDPKDVSGRTVDYIIAGGGLTGLTTAARLTENPNISVLVIESGSYESDRGPIIEDLNAYGDIFGSSVDHAYETVELATNNQTALIRSGNGLGGSTLVNGGTWTRPHKAQVDSWETVFGNEGWNWDNVAAYSLQAERARAPNAKQIAAGHYFNASCHGVNGTVHAGPRDTGDDYSPIVKALMSAVEDRGVPTKKDFGCGDPHGVSMFPNTLHEDQVRSDAAREWLLPNYQRPNLQVLTGQYVGKVLLSQNGTTPRAVGVEFGTHKGNTHNVYAKHEVLLAAGSAVSPTILEYSGIGMKSILEPLGIDTVVDLPVGLNLQDQTTATVRSRITSAGAGQGQAAWFATFNETFGDYSEKAHELLNTKLEQWAEEAVARGGFHNTTALLIQYENYRDWIVNHNVAYSELFLDTAGVASFDVWDLLPFTRGYVHILDKDPYLHHFAYDPQYFLNELDLLGQAAATQLARNISNSGAMQTYFAGETIPGDNLAYDADLSAWTEYIPYHFRPNYHGVGTCSMMPKEMGGVVDNAARVYGVQGLRVIDGSIPPTQMSSHVMTVFYAMALKISDAILEDYASMQ

SEQ ID NO: 2 - protein, 583 aa, synthetic construct
SNGIEASLLTDPKDVSGRTVDYIIAGGGLTGLTTAARLTENPNISVLVIESGSYESDRGPIIEDLNAYGDIFGSSVDHAYETVELATNNQTALIRSGNGLGGSTLVNGGTWTRPHKAQVDSWETVFGNEGWNWDNVAAYSLQAERARAPNAKQIAAGHYFNASCHGVNGTVHAGPRDTGDDYSPIVKALMSAVEDRGVPTKKDFGCGDPHGVSMFPNTLHEDQVRSDAAREWLLPNYQRPNLQVLTGQYVGKVLLSQNGTTPRAVGVEFGTHKGNTHNVYAKHEVLLAAGSAVSPTILEYSGIGMKSILEPLGIDTVVDLPVGLNLQDQTTATVRSRITSAGAGQGQAAWFATFNETFGDYSEKAHELLNTKLEQWAEEAVARGGFHNTTALLIQYENYRDWIVNHNVAYSELFLDTAGVASFDVWDLLPFTRGYVHILDKDPYLHHFAYDPQYFLNELDLLGQAAATQLARNISNSGAMQTYFAGETIPGDNLAYDADLSAWTEYIPYHFRPNYHGVGTCSMMPKEMGGVVDNAARVYGVQGLRVIDGSIPPTQMSSHVMTVFYAMALKISDAILEDYASMQ

SEQ ID NO: 3 - protein, 730 aa, synthetic construct
MRHFWLLPAVAGIAGAQCPYLSGEMSFTQEQDNAGDTIEVTEQPIDNTLYVNDTGSYMTTDFGTPISDQTSLKAGPRGPTLLEDFIFRQKLQRFDHERVPERVVHARGAGAYGTFKSYADWSNVTAADFLSANDKETPMFCRFSTVVGFRGSVDTARDVHGHACRFYTDEGNYDIVGINFAPFFIQDAIQFPDLVHAIKPMPNNEIPQAATAHTSAWDFFSQQSTALHSALWLMSGNGIPRSFRHMNGYGVHSFRFVAANGTSKVVRYRWKSQQGVASLVWDEAQAAAGKNSDYHRQDLYNAIANGHYPKYELQAQIMDEADMLRFGFDLLDPTKLVPEEVVPYTPLGMMELNANPTNYFAEVEQAGFQPGHVVPGIDFTDDPLLQGRLFSYLDTQLTRHGGPNFEQIPVNRPRKPVHNNNRDGFGQQQIPTNNWAYTPNSMSNGYPMQANQTQGHGFFTAPYRYASGHLVRQTSPTFNDHWSQPAMFWNSLIPAEQQMVVNAIVFENSKVNSPHVRKNVVNQLNMVNNNLAVRVARGLGLDEPSPNPTYYTSNKTSNVGTFGKPLLSIEGLQVGFLASNSHPESIKQGQAMAAQFSAAGVDLNIVTEAYADGVNTTYALSDAIDFDALIIADGVQSLFASPALANQMNSTATSTLYPPARPFQILVDSFRYGKPVAAVGSGSVALKNAGIDSSRSGVYTGSSETTEKIAKEVLEGLYTFRFVDRFALDE

SEQ ID NO: 4 - protein, 714 aa, synthetic construct
QCPYLSGEMSFTQEQDNAGDTIEVTEQPIDNTLYVNDTGSYMTTDFGTPISDQTSLKAGPRGPTLLEDFIFRQKLQRFDHERVPERVVHARGAGAYGTFKSYADWSNVTAADFLSANDKETPMFCRFSTVVGFRGSVDTARDVHGHACRFYTDEGNYDIVGINFAPFFIQDAIQFPDLVHAIKPMPNNEIPQAATAHTSAWDFFSQQSTALHSALWLMSGNGIPRSFRHMNGYGVHSFRFVAANGTSKVVRYRWKSQQGVASLVWDEAQAAAGKNSDYHRQDLYNAIANGHYPKYELQAQIMDEADMLRFGFDLLDPTKLVPEEVVPYTPLGMMELNANPTNYFAEVEQAGFQPGHVVPGIDFTDDPLLQGRLFSYLDTQLTRHGGPNFEQIPVNRPRKPVHNNNRDGFGQQQIPTNNWAYTPNSMSNGYPMQANQTQGHGFFTAPYRYASGHLVRQTSPTFNDHWSQPAMFWNSLIPAEQQMVVNAIVFENSKVNSPHVRKNVVNQLNMVNNNLAVRVARGLGLDEPSPNPTYYTSNKTSNVGTFGKPLLSIEGLQVGFLASNSHPESIKQGQAMAAQFSAAGVDLNIVTEAYADGVNTTYALSDAIDFDALIIADGVQSLFASPALANQMNSTATSTLYPPARPFQILVDSFRYGKPVAAVGSGSVALKNAGIDSSRSGVYTGSSETTEKIAKEVLEGLYTFRFVDRFALDE